(19) United States
(12) Patent Application Publication
(10) Pub. No.: US 2002/0031492 A1
(43) Pub. Date: Mar. 14, 2002

(54) Characterizing a brain tumor

(76) Inventor: Waldemar Debinski, Hershey, PA (US)

Correspondence Address: Stanley A. Kim, Akerman, Senterfitt & Eidson, P.A., 222 Lakeview Avenue, Suite 400, P.O. Box 3188, West Palm Beach, FL, 33402-3188, US

(21) Appl. No.: 09/894,609

(22) Filed: Jun. 28, 2001

Related U.S. Application Data
(63) Continuation-in-part of application No. 08/706,207, filed on Aug. 30, 1996.
Application No. 08/706,207 is a continuation-in-part of application No. 08/404,685, filed on Mar. 15, 1995, now Pat. No. 5,614,191.
Application No. 08/706,207 is a continuation-in-part of application No. 09/226,794, filed on Jan. 7, 1999.
(60) Provisional application No. 60/215,623, filed on Jun. 30, 2000.

(51) Int. Cl. G01N 33/574; A61K 38/20

(52) U.S. Cl. 424/85.2; 435/7.23

(57) ABSTRACT

A brain tumor is classified by type or grade includes the steps by quantifying the expression of an IL-13 receptor in a sample of the tumor.

### CROSS REFERENCE TO RELATED APPLICATIONS

The present application is a continuation-in-part of U.S. patent application Ser. No. 08/706,207 filed Aug. 30, 1996 which is a continuation-in-part of U.S. patent application Ser. No. 08/404,685 filed Mar. 15, 1995. The present application is also a continuation-in-part of U.S. patent application 09/226,794 filed Jan. 7, 1999. The benefit of the foregoing applications and also that of U.S. provisional application Ser. No. 60/215,623 filed Jun. 30, 2000 is claimed

### STATEMENT AS TO FEDERALLY SPONSORED RESEARCH

This invention was made with Government support under grant number R01 CA74145 awarded by the Public Health Service. The Government may have certain rights in the invention.

### FIELD OF THE INVENTION

The invention relates generally to the fields of pathology, medicine, and neurooncology. More particularly, the invention relates to the use of interleukin-13 (IL-13) binding as a marker for diagnosing the type and/or grade of a brain tumor, and for assessing the prognosis of a patient having a brain tumor.

### BACKGROUND

The identification of tumor-associated cellular markers has proven useful for diagnosing various tumors and assessing the prognosis of patients with tumors. Cellular markers that occur on the plasma membrane or in a membrane receptor are particularly useful. Antibodies specific for tumor cell markers or ligands that bind specifically to a tumor cell receptor have been successfully used in diagnostics, including both the characterization of excised tissue samples and in vivo imaging.

Numerous different brain tumors are known. For example, several types of brain tumors known as gliomas originate from glial tissue. Within this set of tumors are astrocytomas, brain stem gliomas, ependymomas, and oligodendogliomas. Astrocytomas originate from star-shaped cells termed astrocytes; brain stem gliomas originate in the brain stem; ependymomas originate in the lining of the ventricles or spinal cord; and oligodendrogliomas arise from myelin-producing cells. Brain tumors may also be of non-glial origin. Such non-glial tumors include medulloblastomas, meningiomas, Schwannomas, craniopharyngiomas, germ cell tumors, pineal region tumors, and secondary brain tumors.

Brain tumors are often referred to by grade (grades I-IV), a subjective categorization of a tumor based on the microscopic appearance of its cells. The cells of a high grade tumor (e.g., grade IV) have a more abnormal appearance than cells of a low grade (e.g., grade I) tumor. Cells from grade II and grade III tumors have an appearance intermediate between grades I and IV. Tumors are accorded a grade in order to provide an objective measurement of the seriousness of the disease in a patient with the tumor. Higher grade tumors are generally more malignant, while lower grade tumors are generally less malignant. For example, a grade I astrocytoma is less malignant than a grade II astrocytoma which is less malignant than a grade III (or anaplastic) astrocytoma. The most malignant astrocytoma is a grade IV astrocytoma also known as glioblastoma multiforme (GBM).

It is important to know the type and grade of tumor a patient is suffering from in order to decide the most appropriate treatment to the patient. For example, high grade gliomas such as anaplastic astrocytomas and GBMs grow quickly and infiltrate surrounding tissue easily. In comparison, meningiomas grow much more slowly and with less infiltration. Because of these characteristics, anaplastic astrocytomas and GBMs typically demand more immediate and aggressive treatment than do meningiomas. Thus, methods for determining the type and grade of a brain tumor provide information that is often critical in selecting a course of treatment.

Conventionally, brain tumors are diagnosed by imaging. For example, brain tumors can be detected in situ as abnormal growths by angiography, computerized tomography, and/or magnetic resonance imaging. In some cases, the information provided by imaging may be inadequate to determine the type and/or grade of brain tumor a patient has. To further characterize a brain tumor, the tumor may be biopsied so that a trained pathologist can microscopically examine a section of the biopsied sample to determine the type and grade of the tumor. Such a histopathological examination involves a certain degree of subjectivity on the part of the pathologist. While certain types of tumors may be clearly distinguishable based on microscopic appearance, other are less so. For example, while the prognosis of a patient suffering from a high grade astrocytoma may be much more bleak than that of a patient suffering from a low grade astrocytoma, the histopathological appearance of biopsy samples from the two grades of tumors may be difficult to differentiate and dependent on subjective judgment.

Complicating this, low grade tumors may progress to high grade tumors. Unfortunately, conventional histopathology techniques often do not provide definitive guidance as to which low grade tumors will progress to high grade and which will not.

Thus new methods for differentiating brain tumor types and grades and for providing guidance as to which low grade tumors will progress to high grade would be valuable for assessing the prognosis of a brain tumor, and for determining the most appropriate course of treatment for a brain tumor patient.

### SUMMARY

The invention relates to the discovery that interleukin-13 (IL-13) binding can be used to characterize and distinguish among different types and grades of brain tumors. In the experiments described herein, almost all surgical specimens of a series of 20 human glioblastoma multiformes (GBMs) were determined to over-express specific binding sites for ¹²⁵I-labeled human IL-13 (hIL-13) in situ. This was confirmed in other experiments on samples from over 60 GBMs, where the vast majority of GBMs showed specific binding of labeled IL-13. In comparison, low-grade gliomas (grades I and II) were found to express IL-13 binding sites much more sporadically than did grade III or IV gliomas. Thus, this new finding suggests that the appearance of detectable binding sites for IL-13 accompanies the progression of low- to high-grade gliomas.

IL-13 binding was also assessed in other gliomas and in non-glial origin brain tumors. In these studies, oligodendrogliomas were found to express IL-13 binding sites when the tumor was anaplastic. Surprisingly, pilocytic astrocytomas were also found to possess IL-13 binding sites. In contrast, IL-13 receptor expression was not detected in the non-glial origin brain tumors examined, including secondary brain tumors (metastases) and those tumors of neural or mesodermal origin. Based on the foregoing, the present discovery provides methods and compositions for diagnosing the type and/or grade of a brain tumor, and for assessing the prognosis of a patient having a brain tumor.

Accordingly, in one aspect the invention features a method of classifying a brain tumor by type or grade. This method includes the steps of: (a) providing a brain tumor sample; (b) quantifying the expression of an IL-13 receptor in the sample; and (c) correlating the quantity of expression of the IL-13 receptor on the sample with a tumor type or tumor grade. In this method, the step of correlating the quantity of expression of the IL-13 receptor on the sample with a characteristic of the tumor can be performed by comparing the amount of IL-13 receptor expressed on the sample with the amount of IL-13 receptor expressed on a second brain tumor sample that has previously been characterized by type and grade.

The invention also features a method of distinguishing a higher-grade brain tumor from a lower-grade brain tumor. This method includes the steps of: providing a brain tumor sample; quantifying the expression of an IL-13 receptor in the sample; and correlating the quantity of expression of the IL-13 receptor on the sample with the grade of the tumor. Higher expression of the IL-13 receptor on the sample indicates increased likelihood that the tumor is a higher grade brain tumor, and lower expression of the IL-13 receptor on the sample indicates increased likelihood that the tumor is a lower grade brain tumor.

Also within the invention is a method of analyzing the prognosis of subject with a brain tumor. This method includes the steps of: (a) providing a sample of tissue isolated from a brain tumor in the subject; (b) quantifying the expression of an IL-13 receptor in the sample; and (c) correlating the quantity of expression of the IL-13 receptor on the sample with the prognosis of the tumor in the subject. Higher expression of the IL-13 receptor on the sample correlates with increased likelihood of a poor prognosis, and lower expression of the IL-13 receptor on the sample correlates with decreased likelihood of a poor prognosis.

In the methods of the invention, the IL-13 receptor can be the restrictive form of IL-13 receptor that does not specifically bind IL-4. Additionally, the step of providing the brain tumor sample can include surgically removing at least a portion of a brain tumor from a human patient. The step of quantifying the expression of an IL-13 receptor in the sample can performed by contacting the sample with a probe that specifically binds an IL-13 receptor and then measuring the amount of the probe that binds the sample. The probe can be, e.g., IL-13 (e.g., human IL-13), a fragment of IL-13 that specifically binds the IL-13 receptor, a mutant form of IL-13 that specifically binds the IL-13 receptor, or an antibody that specifically binds the IL-13 receptor. The probe can be conjugated with a detectable label such as a radioactive label, an enzyme, a fluorescent label, or a radio-opaque label.

In another aspect, the invention features a kit for classifying a brain tumor by type or grade. The kit includes a probe that specifically binds an IL-13 receptor; and instructions for using the kit to classify a brain tumor by type or grade.

As used herein, “bind,” “binds,” or “interacts with” means that one molecule recognizes and adheres to a particular second molecule in a sample, but does not substantially recognize or adhere to other structurally unrelated molecules in the sample. Generally, a first molecule that “specifically binds” a second molecule has a binding affinity greater than about 10⁵ to 10⁶ liters/mole for that second molecule.

By the term “antibody” is meant any antigen-binding peptide derived from an immunoglobulin. The term includes polyclonal antisera, monoclonal antibodies, fragments of immunoglobulins produced by enzymatic digestion (e.g., Fab fragments) or genetic engineering (e.g., sFv fragments).

When referring to a protein, the term “mutant” means a modified version of the native protein. A native protein is one found in nature. A protein may be modified by amino acid substitution, deletion, addition, permutation (e.g., circular permutation), etc. “Functional” mutants retain a biological characteristic of the native protein (e.g., the capability of binding of a ligand or producing an enzymatic activity), whereas “non-functional” mutants have lost a biological characteristic.

Unless otherwise defined, all technical terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions will control. In addition, the particular embodiments discussed below are illustrative only and not intended to be limiting.

### DETAILED DESCRIPTION

The invention encompasses compositions and methods for diagnosing the type and/or grade of a brain tumor, and for assessing the prognosis of a patient having a brain tumor. The below described preferred embodiments illustrate adaptations of these compositions and methods. Nonetheless, from the description of these embodiments, other aspects of the invention can be made and/or practiced based on the description provided below.

Biological Methods

Methods involving conventional biological techniques are described herein. Such techniques are generally known in the art and are described in detail in various methodology treatises. For example, molecular biology techniques are described in Molecular Cloning: A Laboratory Manual, 2nd ed., vol. 1-3, ed. Sambrook et al., Cold Spring Harbor Laboratory Press, Cold Spring Harbor, N.Y., 1989; and Current Protocols in Molecular Biology, ed. Ausubel et al., Greene Publishing and Wiley-Interscience, New York, 1992 (with periodic updates). Immunological methods (e.g., preparation of antigen-specific antibodies, immunoprecipitation, and immunoblotting) are described, e.g., in Current Protocols in Immunology, ed. Coligan et al., John Wiley & Sons, New York, 1991; and Methods of Immunological Analysis, ed. Masseyeff et al., John Wiley & Sons, New York, 1992.

Brain Tumor Samples

Methods within the invention include a step of providing a sample of tissue isolated from a brain tumor in a subject. The subject from which the sample is taken will generally be a patient having a brain tumor, although the subject can also be a non-human animal such as a mammal (e.g., dogs, cats, goats, sheep, cows, horses, etc.) having a brain tumor. For laboratory experiments, the subject may be an animal (e.g., a rodent such as an athymic or SCID mouse or rat) into which a tumor has been created such as by xenografting human brain tumor cells. A sample of the tumor can be isolated from a subject by any conventional means. For example, a biopsy of a brain tumor in a human patient can be obtained by known surgical methods. See e.g., Greenberg, M., Handbook of Neurosurgery 5th Ed., Thieme Medical Pub., 2000; Lindsay K. and I. Bone, Neurology and Neurosurgery Illustrated 3rd Ed., Churchill Livingstone, 1997.

Quantifying the Expression of an IL-13 Receptor in a Sample

Methods of the invention also include a step of quantifying the expression of an IL-13 receptor on a brain tumor tissue sample. Numerous methods for characterizing receptor expression on a cell or tissue sample are known. Typically, these methods employ a probe that specifically binds the receptor of interest. The cell or sample is contacted with the probe under conditions that allow the probe to specifically bind to any of the particular receptors on the cells or tissue. Binding of the probe is then quantified as an indication of the amount of receptor on the cells or tissue. To facilitate this, the probe can feature a detectable label such as a radioactive, enzymatic, fluorescent, or radio-opaque (e.g., gold particle) label.

Preferred examples of probes that can be used to quantify IL-13 receptor expression include IL-13 itself (or fragments or mutants thereof that retain the ability to specifically bind the IL-13 receptor) and antibodies (e.g., monoclonal or polyclonal antibodies or fragments thereof) that specifically bind an IL-13 receptor. As “shared” receptors that bind both IL-13 and interleukin 4 (IL-4) are known, a particularly preferred probe is one that detects an IL-13 receptor that does not bind IL-4 (i.e., an IL-13 restrictive receptor), e.g., the IL-13 receptor alpha2 chain. Several mutants of IL-13 that bind the IL-13 restrictive receptor but not the shared receptor are known. See, e.g., International Patent Application Number WO0125282. In addition, where IL-13 is used as a probe, to prevent undesired binding to a shared receptor, the cells or samples being analyzed can be pre-incubated with unlabeled IL-4 as described in U.S. patent application Ser. No. 08/706,207.

Any suitable method for quantifying the amount of a receptor in a sample may be used in the invention. Well known conventional methods that use a probe that binds to a protein receptor include: immunohistochemistry, enzyme-linked immunosorbent assay (ELISA), radioimmunoassay (RIA), direct or indirect immunofluoroscence analysis (e.g., fluorescence microscopy or flow cytometry), and Western blotting. For tissue sections, a preferred method for quantifying the amount of IL-13 receptor in the sample includes the steps of contacting a tissue section with a radiolabeled probe that specifically binds an IL-13 receptor (e.g., 125-I labeled IL-13), and then measuring the amount of radioactivity associated with the section, e.g., by autoradiography. For cells in solution, a preferred method for quantifying the amount of IL-13 receptor in the sample is direct or indirect immunofluoroscence analysis using either a fluorescently labeled antibody that binds an IL-13 receptor, or fluorescently labeled IL-13. For tissue or cells that may be damaged, preferred methods for quantifying the amount of IL-13 receptor in the sample are Western blotting, ELISA, and RIA.

In some cases, the amount of IL-13 expressed by a cell or tissue sample can be approximated by measuring the amount of mRNA encoding an IL-13 receptor in the sample. Numerous methods for measuring the amount of mRNA in a cell or tissue sample are known. For example, quantitative PCR analysis or Northern blotting could be used.

Correlating the Quantity of IL-13 receptor Expression with the Type and Grade of Tumor.

Various methods of the invention include a step of correlating the amount of IL-13 receptor on a cell or tissue sample with the type and/or grade of tumor that the sample was isolated from. Typing and grading of brain tumors is described, e.g., in Fletcher, D. M., Diagnostic Histopathology of Tumors 2nd Ed., Churchill Livingstone, 2000; and McLendon et al., Pathology of Tumors of the Central Nervous System: A Guide to Histologic Diagnosis, Edward Arnold, 2000. As set forth below in the Examples section, certain types and grades of brain tumors are characterized by certain levels of IL-13 receptor expression (as measured by IL-13 binding). For example, most GBMs and grade III gliomas bind IL-13, whereas little or no IL-13 binding was present in low grade gliomas, medulloblastomas or meningiomas. Thus, among glial-derived tumors, higher expression of the IL-13 receptor on the sample appears to correlate with increased likelihood that the tumor is a higher grade brain tumor, while lower expression of the receptor correlates with increased likelihood that the tumor is a lower grade brain tumor.

Based on the foregoing, the invention also provides a method of correlating the quantity of IL-13 receptor expression on a sample of a brain tumor with the prognosis of the subject with the tumor. For example, for gliomas, in some cases, higher expression of the IL-13 receptor on the sample generally will correlate with a poor prognosis of the patient, while lower expression will correlate with a better prognosis.

Kits

The invention also provides kits for diagnosing the type and/or grade of a brain tumor. The kit includes a probe that specifically binds to an IL-13 receptor, a means for detecting the probe (e.g., a detectable label that is associated with the probe or can be caused to bind the probe), and printed instructions for using the kit. The kit can also include other components to assist in quantifying the amount of IL-13 receptor expression in a sample. Such other components might include a substrate to which the cell or tissue sample can be immobilized, e.g., a glass slide or a microtiter plate; or reagents for visualizing the detectable label.

### EXAMPLES

Exemplary methods and compositions that illustrate several aspects of the invention are described below (see also Debinski et al., J. Neuro-Oncol. 48:103, 2000).

### Example 1

Materials and Methods

Production and purification of recombinant proteins. E. coli BL21 (λDE3) cells were transformed with plasmids of interest and cultured in LB Broth (GIBCO/Life Technologies). Procedures for recombinant protein isolation and purification have been previously described (Debinski et al., J. Biol. Chem. 270: 16775-16780, 1995; Debinski et al., Nature Biotech. 16: 449-453, 1998).

Autoradiography. Recombinant hIL-13, EGF, and monoclonal antibody (MAb) HB21 were labeled with ¹²⁵I by using the IODO-GEN reagent (Pierce) according to the manufacturer's instructions. Brain tumor samples were obtained from patients undergoing surgical decompression at Penn State University and University of Alabama at Birmingham Medical Centers. There were 82 patients evaluated in this study, with 41 females and 37 males, age 1 to 81 years (4 without gender identification). Serial tissue sections were cut (10 μm) on a cryostat, thaw-mounted on chrome-alum coated slides, and stored at −80° C. until analyzed (Debinski et al., Clin. Cancer Res. 5: 985-990, 1999). To observe binding distribution of ¹²⁵I-ligands, sections were incubated exactly as described (id.). After drying, labeled sections were apposed to Kodak autoradiography film at −70° C. for 1 to 3 days on average. For autoradiography on cultured cells, 5×10⁴ cells were placed on a sterile glass slide in a small volume of media and allowed to attach. The cells were maintained overnight at 37° C. The slides were then washed in two changes of 0.1 M PBS and fixed with ethanol. The slides were rinsed again with 0.1 M PBS and processed for autoradiography. Autoradiographic images were scanned using Agfa's Arcus II scanner (Ridgefield Park, N.J.) at 675 pixels/in². The images were processed using Paint Shop Pro JASC Software (Minnetonka, Minn.).

### Example 2

Results

Low-grade glioma tissue staining. To demonstrate the presence of binding sites for IL-13, EGF, and Tf in clinical specimens of brain tumors in situ, autoradiographic analysis using appropriate radiolabeled ligands in tissues derived primarily from GBM patients was performed (Debinski et al., Clin. Cancer Res. 5: 985-990, 1999; Debinski et al., Int. J. Oncol. 15:481, 1999). These studies provided evidence for the presence of IL-4-independent binding sites for IL-13 in a vast majority of patients with GBM. Binding sites of this characteristic were also found on a majority of established GBM cell lines (Debinski et al., J. Biol. Chem. 271:428, 1996). To further analyze phenotypic appearance of other than high-grade gliomas with regard to the expression of IL-13 binding sites, autoradiography was performed on samples of multiple brain tumors using ¹²⁵I-radiolabeled IL-13, EGF, and a monoclonal antibody against human transferrin receptor (TfR), HB21. The study was designed to be done on same-patient contiguous tissue sections of the same piece of tumor, whenever possible, for all the ligands.

Eleven low-grade gliomas showed little evidence for ¹²⁵I-hIL-13 specific binding by most of the samples. Only fibrillary low-grade glioma and two grade II samples showed signs of radiolabeled IL-13 specific binding to various degrees. This binding, of interest, was mainly IL-4-independent as an excess of unlabeled hIL-13, and not IL-4, competed for the binding of ¹²⁵I-hIL-13 in those tumor specimens. Also, a minority of the low-grade glioma studied expressed EGF binding sites, however, the sample of mixed oligo #14 was extremely enriched in this receptor. The binding sites for anti-transferrin receptor antibody, HB21, were present uniformly among low-grade gliomas, although the intensity of the binding was relatively low. Thus, only 3/11 low-grade gliomas exhibited IL-4-independent binding sites for IL-13.

High-grade glioma tissue staining. Demonstrating that low-grade gliomas are only sporadic expressors of IL-13 binding sites, further high-grade glioma specimens were analyzed. Autoradiography was performed on five available specimens of grade III astrocytomas. All showed clearly positive binding of radiolabeled IL-13. Except for one specimen, this binding was mostly IL-4-independent. In addition to this group of grade III astrocytomas, autoradiography was performed on another group of 20 new specimens of grade IV astrocytomas (i.e., GBMs). GBM bound radiolabeled IL-13 uniformly and mainly in an IL-4-independent manner. Specimens of 3 recurrent GBMs showed a similar pattern of IL-13 binding. In other experiments on more than 40 tissue specimens of GBM similar IL-13-binding results were obtained. Thus, there is a profound difference between low- and high-grade gliomas in terms of the presence of significant amounts of IL-13 binding sites. Only a small subgroup of low-grade gliomas over-express IL-13 binding sites while high-grade glioma ubiquitously demonstrate high expression levels of these sites.

Other than low- or high-grade glioma astrocytic tumors staining. In addition to the foregoing, several other forms of astrocytomas, such as oligodendrogliomas, ependymomas, and pilocytic astrocytomas were examined. Among oligodendrogliomas, an anaplastic form of these tumors showed readily positive staining for ¹²⁵I-IL-13 and those binding sites proved to be IL-4-independent. However, the presence of IL-13 binding was not detected in two samples of differentiated oligodendrogliomas. Ependymomas also appeared to be phenotypically silent for IL-13 binding. In a very unexpected development, all six pilocytic astrocytomas tested exhibited a clear-cut presence of IL-13 binding sites of a restrictive in character, i.e. IL-4-independent.

Binding of IL-13 to brain tumors of other than glial origin. None of the four medulloblastoma brain tissue samples and one ganglioglioma tested showed appreciable affinity for ¹²⁵I-hIL-13. The examined tissues showed variable retention of ¹²⁵I-hIL-13, which was not changed in the presence of an excess of hIL-13. However, the DAOY medulloblastoma cell line obtained from ATCC bound ¹²⁵I-IL-13 very densely and specifically for IL-13, and not for IL-4—a result in line with the previous observation that DAOY cells are extremely responsive to the IL-13-based cytotoxins. Neither medulloblastomas nor gangliogliomas demonstrated significant specific ¹²⁵I-EGF binding. However, the receptor for transferrin was present in all the samples tested. Among other brain tumors, two gliosarcomas were positive for an IL-4-independent receptor for IL-13, but not acoustic neuroma, choroid plexus papilloma, or rhabdomyosarcoma.

The lack of IL-13 receptors in meningiomas. 20 meningiomas were subjected to autoradiographic analysis. Only two specimens out of 20 showed positivity for IL-13 binding sites. However, at least seven meningioma samples stained for EGFR and practically all of them showed the presence of transferrin receptor. Thus, the binding sites for IL-13 are absent among meningiomas.

Metastases to brain and IL-13 binding. 12 brain tumors, identified as metastases to the brain, were obtained. Only four tumor samples showed binding sites for ¹²⁵I-hIL-13. Three of these were adenocarcinomas originating from the lung, and one was a renal cell carcinoma. A similar percentage of studied metastases (4/12) showed detectable binding for EGF. Again, the pattern of staining for the TfR by radiolabeled antibody HB21 was comparable to other brain tumors, i.e. it was present in virtually all tumors with differing degree of its density.

Other Embodiments

This description has been by way of example of how the compositions and methods of invention can be made and carried out. Those of ordinary skill in the art will recognize that various details may be modified in arriving at the other detailed embodiments, and that many of these embodiments will come within the scope of the invention. Therefore, to apprise the public of the scope of the invention and the embodiments covered by the invention, the following claims are made.

What is claimed is:

1. A method of classifying a brain tumor by type or grade, the method comprising the steps of:
(a) providing a brain tumor sample;
(b) quantifying the expression of an IL-13 receptor in the sample; and
(c) correlating the quantity of expression of the IL-13 receptor on the sample with a characteristic of the tumor, the characteristic being selected from the group consisting of tumor type and tumor grade.

2. The method of claim 1, wherein the IL-13 receptor is the restrictive form of IL-13 receptor that does not specifically bind IL-4.

3. The method of claim 1, wherein the step (a) of providing the brain tumor sample comprises surgically removing at least a portion of a brain tumor from a human patient.

4. The method of claim 1, wherein the step (b) of quantifying the expression of an IL-13 receptor in the sample is performed by contacting the sample with a probe that specifically binds an IL-13 receptor and then measuring the amount of the probe that binds the sample.

5. The method of claim 4, wherein the probe is selected from the group consisting of IL-13, a fragment of IL-13 that specifically binds the IL-13 receptor, and a mutant form of IL-13 that specifically binds the IL-13 receptor.

6. The method of claim 5, wherein the probe is selected from the group consisting of human IL-13 and a human IL-13 mutant that specifically binds an IL-13 receptor.

7. The method of claim 4, wherein the probe is an antibody that specifically binds the IL-13 receptor.

8. The method of claim 4, wherein the probe is conjugated with a detectable label.

9. The method of claim 8, wherein the detectable label is selected from the group consisting of a radioactive label, an enzyme, a fluorescent label, and a radio-opaque label.

10. The method of claim 1, wherein the step (c) of correlating the quantity of expression of the IL-13 receptor on the sample with a characteristic of the tumor comprises comparing the amount of IL-13 receptor expressed on the sample with the amount of IL-13 receptor expressed on a second brain tumor sample that has previously been characterized by type and grade.

11. A method of distinguishing a higher-grade brain tumor from a lower-grade brain tumor, the method comprising the steps of:
(a) providing a brain tumor sample;
(b) quantifying the expression of an IL-13 receptor in the sample; and
(c) correlating the quantity of expression of the IL-13 receptor on the sample with the grade of the tumor, wherein higher expression of the IL-13 receptor on the sample indicates increased likelihood that the tumor is a higher grade brain tumor, and wherein lower expression of the IL-13 receptor on the sample indicates increased likelihood that the tumor is a lower grade brain tumor.

12. The method of claim 11, wherein the IL-13 receptor is the restrictive form of IL-13 receptor that does not specifically bind IL-4.

13. The method of claim 11, wherein the step (a) of providing the brain tumor sample comprises surgically removing at least a portion of a brain tumor from a human patient.

14. The method of claim 11, wherein the step (b) of quantifying the expression of an IL-13 receptor in the sample is performed by contacting the sample with a probe that specifically binds an IL-13 receptor and then measuring the amount of the probe that binds the sample.

15. The method of claim 14, wherein the probe is selected from the group consisting of IL-13, a fragment of IL-13 that specifically binds the IL-13 receptor, and a mutant form of IL-13 that specifically binds the IL-13 receptor.

16. The method of claim 15, wherein the probe is selected from the group consisting of human IL-13 and a human IL-13 mutant that specifically binds an IL-13 receptor.

17. The method of claim 14, wherein the probe is an antibody that specifically binds the IL-13 receptor.

18. The method of claim 14, wherein the probe is conjugated with a detectable label.

19. The method of claim 18, wherein the detectable label is selected from the group consisting of a radioactive label, an enzyme, a fluorescent label, and a radio-opaque label.

20. A method of analyzing the prognosis of subject with a brain tumor, the method comprising the steps of:
(a) providing a sample of tissue isolated from a brain tumor in the subject;
(b) quantifying the expression of an IL-13 receptor in the sample; and
(c) correlating the quantity of expression of the IL-13 receptor on the sample with the prognosis of the tumor in the subject, wherein higher expression of the IL-13 receptor on the sample correlates with increased likelihood of a poor prognosis, and wherein lower expression of the IL-13 receptor on the sample correlates with decreased likelihood of a poor prognosis.

21. The method of claim 20, wherein the IL-13 receptor is the restrictive form of IL-13 receptor that does not specifically bind IL-4.

22. The method of claim 20, wherein the step (a) of providing the brain tumor sample comprises surgically removing at least a portion of a brain tumor from the subject, the subject being a human patient.

23. The method of claim 20, wherein the step (b) of quantifying the expression of an IL-13 receptor in the sample is performed by contacting the sample with a probe that specifically binds an IL-13 receptor and then measuring the amount of the probe that binds the sample.

24. The method of claim 23, wherein the probe is selected from the group consisting of IL-13, a fragment of IL-13 that specifically binds the IL-13 receptor, and a mutant form of IL-13 that specifically binds the IL-13 receptor.

25. The method of claim 24, wherein the probe is selected from the group consisting of human IL-13 and a human IL-13 mutant that specifically binds an IL-13 receptor.

26. The method of claim 23, wherein the probe is an antibody that specifically binds the IL-13 receptor.

27. The method of claim 23, wherein the probe is conjugated with a detectable label.

28. The method of claim 27, wherein the detectable label is selected from the group consisting of a radioactive label, an enzyme, a fluorescent label, and a radio-opaque label.

29. A kit for classifying a brain tumor by type or grade, the kit comprising:
a probe that specifically binds an IL-13 receptor; and
instructions for using the kit to classify a brain tumor by a characteristic selected from the group consisting of type and grade.
